(19) United States
(12) Patent Application Publication
(10) Pub. No.: US 2003/0233074 A1
(43) Pub. Date: Dec. 18, 2003

(54) IV access connectors for syringes with needles

(76) Inventor: Jack W. Shields, Santa Barbara, CA (US)

Correspondence Address: Michael G. Petit, Ph.D., P.C., Patent Law Office, P.O. Box 91929, Santa Barbara, CA, 93190-1929, US

(21) Appl. No.: 10/172,581

(22) Filed: Jun. 17, 2002

(51) Int. Cl. A61M 5/32

(52) U.S. Cl. 604/198

(57) ABSTRACT

A connector device for conveying a sterile fluid medication housed within a cylindrical container, such as a syringe or pre-filled cartridge having a sharp hollow needle attached thereto, into a needle-penetrable intravenous (IV) access port on an intravenous IV infusion system. The device enables the transfer of medicament from within the cylindrical container into the IV access port without hazardous exposure of the sharp hollow needle or bacterial contamination of the IV access port. The connector device is a hollow tubular structure having a length, a conical or cylindrical leading axial bore, and a conical trailing axial bore. The leading bore is dimensioned to snugly accept a portion of the infusion limb of the IV access port into a fixed recessed position. The trailing conical bore is dimensioned to accommodate and wedge impact a portion of the cylindrical medication container. When the leading portion of the cylindrical container is wedge impacted into the trailing conical bore and can be advanced no further thereinto, the sharp hollow needle attached thereto penetrates the recessed portion of infusion limb of the IV access port, such that the medication can be transferred from the container into the port. On completion of the transfer, the connector device is removed from the IV access port still containing the wedge impacted cylindrical container whereupon the attached needle remains safely recessed in the leading axial bore of the connector device. The connector device containing the safely recessed needle and wedge impacted cylindrical container, then, become safely disposable as a unit. Compared with needleless IV access ports having on-line recesses prone to bacterial colonization, this connector device maximizes the efficient sterile use of inserted sharp hollow needles at low costs.

### BACKGROUND OF THE INVENTION

1. Field of the Invention

There is an urgent need for making intravenous infusions safer for patients and for healthcare workers (HCWs), such that the risks for common bacterial infections and bloodbome virus infections are minimized. This simple hollow cone minimizes the mutual risks efficiently and cost-effectively.

2. Description of Prior Art

For at least 80 years, the intravenous (IV) infusion of sterile fluids and soluble medications increasingly became a mainstay of medical treatment. During the 1980s, Hepatitis B (HBV) and AIDS (HIV) and, later, hepatitis C (HCV) transmitted via needles were recognized as major public health problems. Recognition spurred increased efforts by the medical profession and government to increase needle safety and sterility of equipment and fluids essential to IV, as well intramuscular and subcutaneous medication therapy. In 1987 “Universal Precautions” issued to minimize cross-infections between patients and HCWs, especially cross infections with bloodbome pathogens, most notably HBV, HIV and HCV. Needle safety was prioritized, along with use of gloves by HCWs. while handling body fluids or needles potentially contaminated with blood. In 1992, Federal Directives recommended use of “needleless systems” insofar as possible, in order to avoid accidental needlesticks to HCWs during the injection of liquid medications into patients. Such directives and precautions were reinforced by Federal legislation on Jan. 18, 2001.

Insofar as patients are concerned, side-effects of “Universal Precautions” in 1987 have been diminution of HCWs handwashing before donning gloves and excessive use of non-sterile examination gloves when touching patients and manipulating equipment used for giving injections and IV infusions. Side effects of the 1992 Federal Directives and 2001 Federal Legislation have been the excessive use of needleless IV access ports on IV infusion systems resulting in excessive numbers of hospital-acquired blood str6am infections (BSIs) with common skin-home bacteria, especially with coagulase-negative Staphylococci (CAS) Staphylococcus aureus (SA). The severity of such BSIs has increased, owing to evolving resistance of CNS, SA and other microbial infections to antibiotics currently available. (See Medical References 1-2,11-12)

Focusing on IV access ports, it should be emphasized first that the term, needleless system is a misnomer. Sharp hollow needles attached to the leading ends of IV infusion systems are customarily needed to access veins or arteries directly, or to insert over-the-needle catheters. In needleless IV access ports currently in common use, hollow needles are needed to fill syringes from vials or ampoules before taking off the needle and replacing it with a blunt cannula, or taking the needle off to leave a filled Luer-Lok syringe attachable to a needleless IV access port. Thus, before use of the port, there are hazards for bare-handed skin bacterial or non-sterile glove contamination of the cannulae, cannula hubs or syringe nozzles. Use of pre-filled cartridges with attached blunt cannulae may avoid some of these hazards. In this case, however, the attached blunt cannula must be supplied with an air-tight scabbard to prevent bacterial contamination, as well as fluid leakage from the filled cartridge before insertion into the needleless infusion port.

Additional hazards of needleless ports are that all embody on-line slits or definitive recesses prone to bacterial colonization when main line fluid refluxes into such on-line recesses under the aegis of main line hydrostatic pressure during the act of blunt cannula or syringe nozzle withdrawal. The refluxed fluid, if colonized by microorganisms from unwashed hands, non-sterile gloves, contaminated cannulae or nozzles, or inadequate sterilization of on-line slits, rim recesses or recessed chambers in the ports, some colonies will be injected IV with repeated use of the same needleless port. Sterile capping of the port between uses, although a deterrent to air- or water-home contamination, does not eliminate this hazard because the cap must be removed before and during each port access. The number of BSIs emanating from such needleless ports can be expected to be proportional to the nutrient composition of the main line infusion fluid, the number of times the port is reused, and the difficulties encountered with sterilizing the on-line slits, rim recesses and chambers proximal to on-line valves. (See Medical References 2-11).

As opposed to the needleless IV access ports, conventional ports accessed by sharp needles are usually capped by latex-free elastomers without rims holding down slit diaphragms or recesses made for insertion of syringe nozzles. Being flat and rounded on the trailing external surfaces, the caps are easy to sterilize with alcohols. povidone iodine or chlorhexadine in varying combinations. The cap substance is penetrable by sharp small bore steel needles which seldom penetrate twice or more through the same track. The cap is held in place on the trailing end of a straight, Y- or T-intermittent infusion limbs connecting to the main stream lines of IV infusion systems. Essentially, the cap serves as a valve which prevents back-flow from the main-line, except when penetrated sufficiently by the bevel and shank of the small bore needle. The leading end of the cap embodies a cylindrical hollow defect that aids insertion of the elastomeric cap during manufacture and minimizes the distance the bevel and shank of the needle must axially penetrate precisely to establish fluid continuity with the main stream. The cap is stabilized outside the intermittent infusion limb by a constricting collar of plastic material which compresses an outside skirt of the cap against the outside trailing end of the infusion limb. Compression of the skirt by the collar usually leaves a bulging trailing end of the cap useful for stabilization of the limb, when the cap is snugly inserted with compression into a cylindrical or fittingly coned leading end of the present invention. For descriptive purposes, but not shown in the drawings, this prior art version of a “standard” or “conventional” IV access. port will be used in the text which follows.

The structural prior art applicable to needleless IV access ports on IV infusion systems is not pertinent to this patent application, albeit the connector is “needleless” in this invention. The connector is designed to safely connect conventional IV access ports with needles attached or attachable to standard syringes and prefilled cartridges. The pertinent prior art is found in U.S. Pat. Nos. 4,998,925; 5,558,649, and 6,245,056.

In U.S. Pat. No. 4,998,925, Al-Sioufi disclosed a port injector comprising a needle recessed in a cylindrical scabbard, the leading end having an expansion fitting over a conventional IV access port and the trailing end having a hub attachable to a syringe. Functionally, the recessed needle can not be used to fill the syringe from a standard vial or ampoule. As opposed to the instant invention, '925 structurally embodies a recessed needle sharp on one end with a trailing hub attachable to a syringe. Similar IV access port injectors incorporating recessed needles and having various means for attachment to the infusion port are in common use.

In U.S. Pat. No. 5,558,649, Shields disclosed a hollow conical syringe/needle shield with a closed apex; an open frustum having an internal diameter greater than the external diameter of a syringe barrel or greatest external diameter of another object with leading sharp parts; and a conical bore therebetween which wedge impacts an inserted syringe barrel or other object of similar maximal diameter, such that the bevel and shank of a syringe-attached needle or sharp parts of the other inserted object are protected in the closed leading end of the hollow cone. The apex of the hollow cone being closed, '649 is not suitable for direct attachment-to an IV access port.

In U.S. Pat. No. 6,245,056, Walker and Shields disclose an IV access port connector/injector comprising a hollow cylinder incorporating a transverse septum permanently holding a safely recessed hollow needle sharp on each end for transferring fluid from a carpule without an attached needle into an IV access port or for inter-connecting two similar ports. The leading end or both ends of the hollow cylinder are biased or flared to facilitate axial insertion of the carpule and one port, or two similar IV access ports. Therefore, lacking a transverse septum and a needle, the instant invention is different.

Although the instant invention incorporates useful features from '925, '649 and '056, it differs essentially in that it is hollow cone, open on each end and without an embodied needle. As such, it is simple, efficient, safe and relatively inexpensive to use with a wide variety of standard needles and syringes or cartridges for-the intended purposes.

3. Additional Notes

The configurations of IV access ports and diameters of their elastomeric needle, penetrable caps made by differing manufactures vary significantly. Also, the external and internal diameters of syringes and pre-filled carpules or cartridges made by differing manufacturers vary. The external diameters of standard 3.0 mL. Luer-Lok syringes closely approximate the external diameters of conventional needle-penetrable infusion port caps. Depending on the volume of fluid contained, e.g. 1.8 mL. in dental carpules and 1.0 mL. to 5.0 mL. in medical cartridges and syringes, external diameters and lengths vary accordingly. Notwithstanding, as shown in the drawings, such variations can be accommodated by sloping of cones and cylindrical parts in the connectors, along with modifications of openings at each end for convenience and stability. In addition, using needle shanks and scabbards of differing lengths will permit safe efficient use of syringes or cartridges with minor differences in barrel sizes. For larger volume IV access port infusions, the interconnection of ports, e.g. piggy back to main line might be preferable, as disclosed in U.S. Pat. No. 6,245,056.

### SUMMARY OF THE INVENTION

I disclose a unique connector having a trailing conical bore that wedge impacts the leading end of the inserted barrel of a syringe or filled cartridge, and a leading cylindrical or conical bore which concentrically grasps the elastomeric cap and intermittent infusion limb of a conventional IV access port, such that the bevel and shank of cartridge or syringe-attached needle penetrates the cap to permit flow of sterile fluid into the port; and such that the exposed bevel and shank of needle without a covering scabbard is continually recessed safely within the leading bore of the connector. Practical features include:

The connector virtually eliminates the patient hazards of needleless IV access ports with built-in on-line slits or recesses prone to bacterial colonization and IV injection of colonies with reuse of the same port

When the sharp needle without a scabbard is confined in a connector supplied sterile, the needle cannot be contaminated by touching a foreign object or touched to produce a needlestick injury to a HCW or a patient.

The connector is efficient and easy to use, because axial alignment of conical, cylindrical and elastomeric parts assures accurate guidance, as well as protection of the of the needle during insertion and after use for the intended purposes.

Because a needle supplied attached to a sterile syringe or a cartridge is protected from exposure by its fitting scabbard before syringe filling and the connector provides a sterile haven for the attached needle from the time of filling until disposal along with the connector, opportunities for touching or being injured by its bevel or shank are minimized, whether or not HCWs wash hands adequately or wear sterile gloves during manipulation.

Being time-efficient during use and not expensive to manufacture, the connector should prove ideal for minimizing cross-infections from patients to HCWs and vice versa during use with conventional IV access ports.

### BRIEF DESCRIPTION OF THE DRAWINGS

FIG. 1 The essential components of a conventional Y-shaped IV access port.

FIG. 2 Essential components of an applicable hollow conical connector.

FIG. 3 Essential components of a pre-filled cartridge useful with the former.

FIG. 4 The cartridge components assembled and filled with liquid medication.

FIG. 5 The filled cartridge inserted into a useful position in the hollow connector.

FIG. 6 The cartridge mated inside the connector to the Y-port via a sharp needle.

FIG. 7 The cartridge contents emptied into the Y-port via the sharp needle.

FIG. 8 The connector/empty cartridge assembly ready for safe disposal after use.

FIG. 9 The essential components of a straight IV access port,

FIG. 10 Essential components of an applicable hollow conical connector.

FIG. 11 Essential components of a standard syringe/needle useful with the former.

FIG. 12 The syringe/needle with needle scabbard removed before filling with liquid.

FIG. 13 The filled syringe/needle ready for insertion into the bore of the connector.

FIG. 14 The inserted syringe mated inside the connector via the needle to the infusion port.

FIG. 15 The syringe contents expelled into the IV access port via the needle.

FIG. 16 The connector/empty syringe and needle assembly ready for safe disposal after intended use and disconnection from the IV access port.

FIGS. 17-21 Alternative configurations of the hollow conical connector for efficient use with pre-filled cartridges and syringes of differing fluid volume capacities and attached needles of differing lengths.

### DESCRIPTION OF PREFERRED EMBODIMENTS

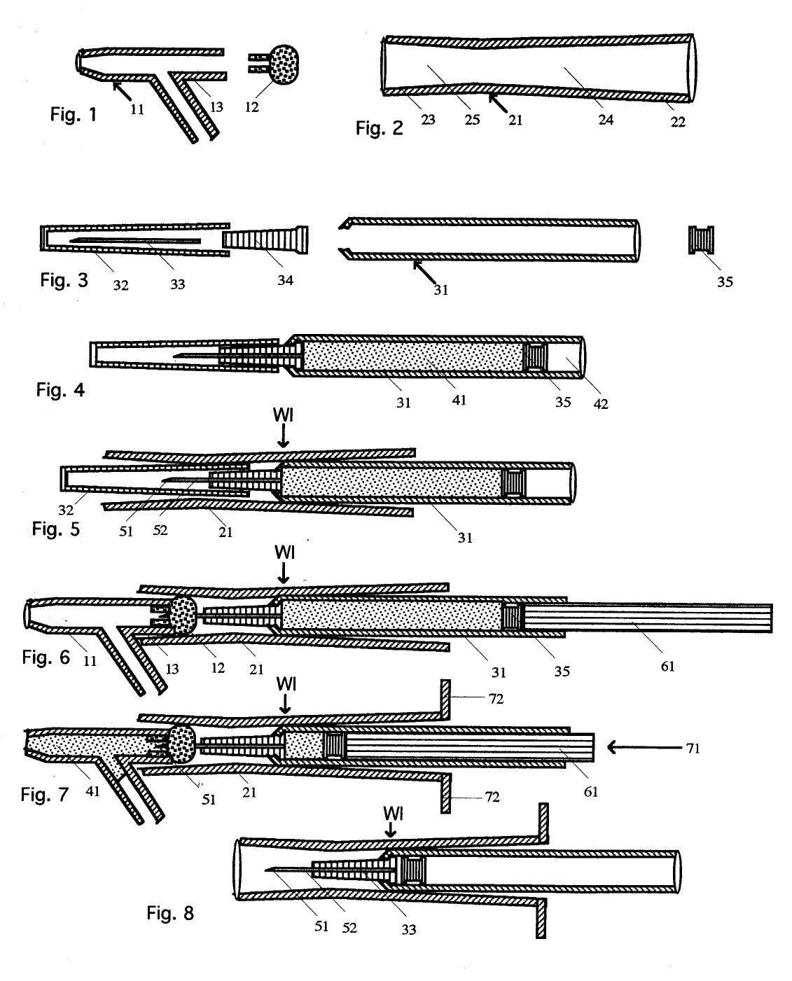

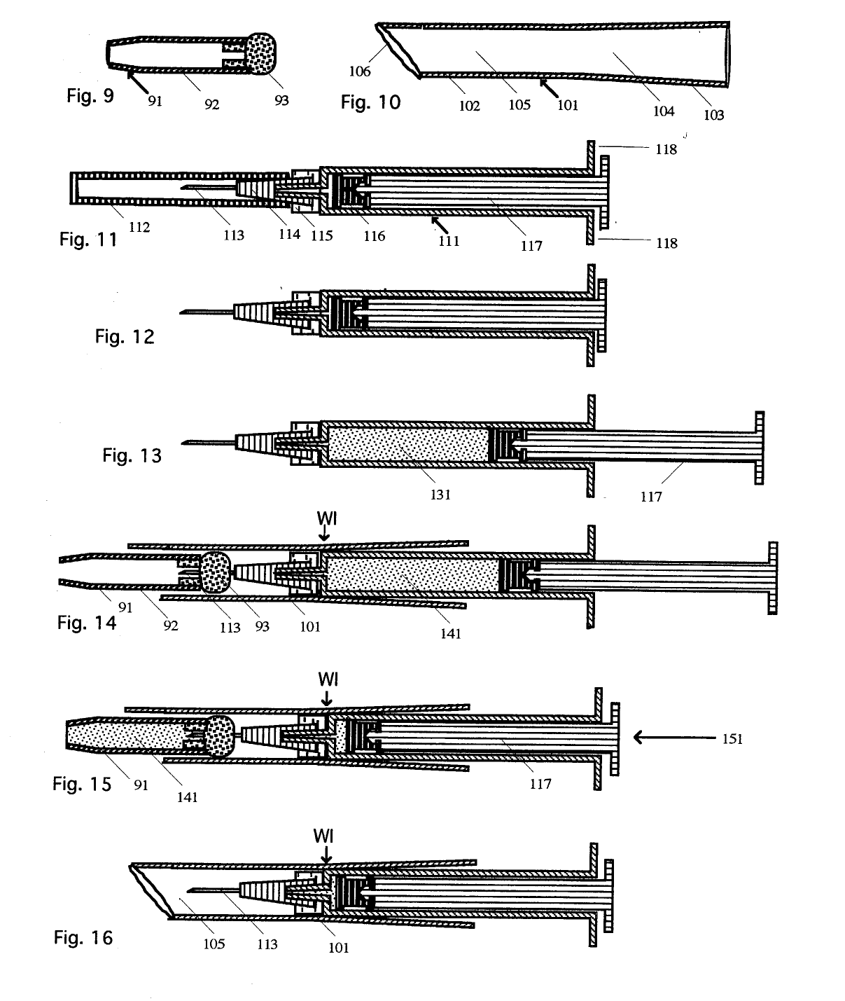

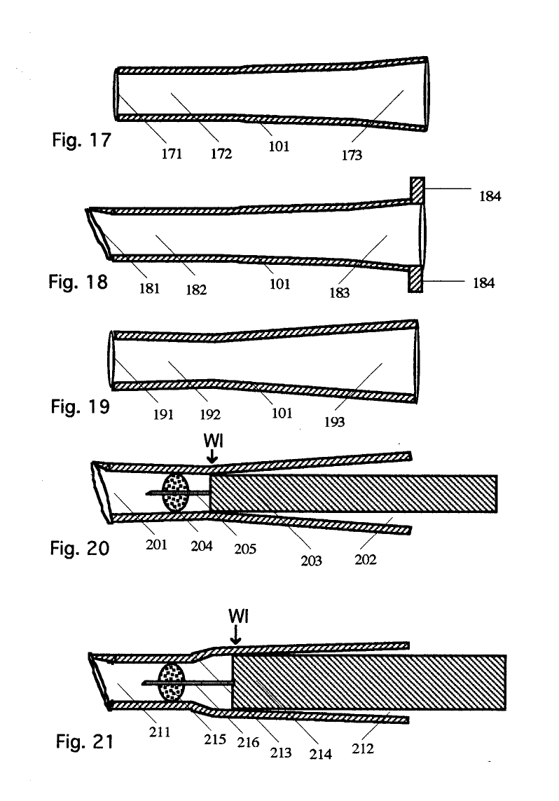

FIGS. 1-8 show a first embodiment wherein a pre-filled cartridge with a permanently attached needle injects a conventional Y-port FIGS. 9-16 show another embodiment wherein a standard Luer-Lok syringe with an attached needle injects a conventional straight infusion port on an IV infusion assembly. FIGS. 17-21 show useful variations in the shape of the connector which enhances safe sterile fluid medication transfer from the hollow cylindrical cartridge or syringe into the IV access ports.

FIG. 1 shows the essential components of a standard Y-shaped access port 11 with a needle-penetrable elastomeric cap 12 on the intermittent infusion limb 13.

FIG. 2 shows a hollow tubular connector 21 with a coned configuration on the trailing 22 and leading 23, ends, such that a conical trailing 24 bore can wedge impact the barrel of an inserted standard cartridge with a permanently attached needle and the conical leading bore 25 can snugly accept the elastomeric cap 13 on the intermittent infusion limb 12 of the IV access port 11 in a position useful for transferring fluid from the cartridge into the Y-port via a needle which is always safely enclosed in the leading bore 25 of the connector 21 during and after intended use. The connector 21 should be optically transparent and preferably less rigid in substance than glass or plastics normally used to make liquid medication-filled hollow cylinders. Polypropylene is a preferable material for making the connector 21, because it is EPA compatible with respect to disposition.

FIG. 3 shows the essential parts of a standard cartridge 31 including from left to right: a needle scabbard 32, sharp hollow needle 33, needle hub 34, glass or plastic hollow cartridge 31 and a cartridge piston 35. As opposed to standard cartridge pistons, the piston does not embody a trailing screw for attaching the leading end of a plunger. This deviation from conventional is because it is not necessary to aspirate fluid from an IV access port before injecting, and dilution by aspirated mainstream fluid might interfere with dosage measurement when fractional doses are critical. As opposed to plastics for cartridge substance, glass is probably preferable, because many liquid medications are better preserved, especially during prolonged storage.

FIG. 4 shows the cartridge parts 31-35 assembled and the cartridge 31 filled with sterile fluid medication 41. An unusual amount of space is left behind the piston 35 in the trailing bore 42 of the cartridge 31. More space is left for several reasons: (a) longer axial space provides better stabilization of an inserted plunger; (b) less axial space in front of the piston allows cartridges having the same dimensions to hold smaller doses and inject single or fractional doses more accurately; and (c) more axial space facilitates each stable insertion of a plunger, and, after withdrawal of the plunger, prevents accidental activation of the piston, especially between fractional doses given over extended periods of time.

FIG. 5 shows the filled cartridge 31 inserted through the trailing bore of the connector 21 to a point of wedge impaction WI, such that the leading bevel 51 and shank 52 of the needle 33 are fixed in a stable central axial position to penetrate the cap 13 on an inserted infusion limb 12 of the Y-port 11. Note that the needle scabbard 32 has not been removed, because removal is not necessary for filling the cartridge. Note, also, that the exposed bevel 51 and shank 52 are unusually short, compared with the length of the scabbard 32. This length disparity allows the user (a) to pull off the scabbard through the leading end of the connector without exposing the bevel and shank before use in the Y-port; (b) to tighten the wedge impaction while doing so; and (c) prevent fluid leakage from the needle bevel until it's the appropriate time for giving the injection.

FIG. 6 shows the pre-filled cartridge 31 inserted into the connector, wedge impacted WI, and with the needle scabbard removed, such that the sharp needle is in a useful and protected position after penetrating the elastomeric cap 13 on the intermittent infusion limb 12 of the Y-port 11. Then, a fitting plunger 61 is inserted into the leading bore of ‘the prefilled cartridge 31 for activating the piston 35 to inject the prescribed dose of liquid medication 41.

FIG. 7 shows the cartridge contents 41 partly emptied into the Y-port 11 by manual pressure exerted on the plunger 61 in the direction of the arrow 71. Note that flanges 72 for finger placement are added optionally to the basic configuration of the connector 21 to facilitate one-handed operation of the plunger for expelling medication from the cartridge into the Y-port Such a modification would prove useful, especially with short cartridges.

FIG. 8 shows the connector/cartridge assembly ready for safe disposal after cartridge emptying, removal of the cartridge plunger and detachment from the Y-port. Note safe recessing of the sharp bevel 51 and shank 52 of needle 33 in the disposable assembly.

FIG. 9 shows a straight IV access port 91 with a single infusion limb 92 having a conventional elastomeric cap 93 on the trailing end.

FIG. 10 shows a hollow tubular connector 101 with a cylindrical configuration on the leading end 102 and a conical configuration on the trailing end 103, such that a conical trailing bore 104 can wedge impact the barrel of an inserted standard syringe with an attached needle; and such that a cylindrical barrel on the leading end can snugly accept the elastomeric cap 93 on the infusion limb 92 on the IV access port 91 in a position useful for transferring sterile fluid from a filled syringe into the port via a needle which is always safely enclosed in the leading bore 105 during and after intended use. The leading end 102 and bore 105 of the connector 101 are cut on a bias 106 to facilitate insertion of the elastomeric cap 93 on the access port 91, more or less like a shoe horn eases insertion of a heel into a tight shoe, Material specifications for the connector 101 are the same as outlined for the connector 21 in FIG. 2.

FIG. 11 shows the essential parts of a standard 3.0 mL Luer-Lok syringe 111 including from left to right: a needle scabbard 112, a sharp hollow needle 113, a needle hub 114, a Luer-Lok 115 which secures the syringe nozzle to the needle hub, a sliding piston 116, a plunger 117 permanently attached to the trailing end of the piston, and trailing flanges 108 for finger placement

FIG. 12 shows the same parts with the needle scabbard removed, such that the syringe can be filled via the needle from a vial, ampoule or other container with sterile liquid medication.

FIG. 13 shows the syringe filled with the liquid medication 131 by retraction of the plunger 117

FIG. 14 shows the filled syringe 141 inserted to a point of wedge impaction WI in the trailing conical bore of the connector 101 and the infusion port 91 inserted into a leading cylindrical bore of the connector to the point where the needle 113 penetrates through the elastomeric cap 92 and the infusion limb is partially stabilized in the leading bore. Because it is not safe to recap a needle with its fitting scabbard after a syringe is filled, the filled syringe 141 with the attached needle 113 should be inserted into the connector 101 immediately after filling and before the infusion port 91 is inserted. Safely confined within the connector by a firm wedge impaction WI of the leading end of the syringe barrel, the assembly of connector over syringe and needle can be transported without contamination or accidental needlestick to the site of actual use on an established IV infusion system.

FIG. 15 shows the fluid 141 in the syringe expelled into the infusion port 91 by force in the direction of the arrow 151 on the plunger 117.

FIG. 16 shows the connector over syringe and needle ready for safe disposal after emptying the syringe and detachment from the infusion port Again, note the safe recessing of the needle 113 in the leading bore 105 of the connector 101.

FIG. 17 shows an alternative connector profile having no bias on the leading end 171, a cylindrical leading bore 172 and a conical trailing bore with a terminal flare 173. This alternative makes it more difficult to insert a conventional IV access port cap into the leading bore, but facilitates the insertion of a cylindrical cartridge or syringe into to the trailing end.

FIG. 18 shows an alternative connector profile having a biased leading end 181, a cylindrical leading bore 182, a flared trailing bore 183 and added terminal flanges 184 for finger placement This profile might prove advantageous, especially for insertion of short pre-filled cartridges containing less than 2.0 mL. of sterile fluid medication for injection into an inserted IV access port.

FIG. 19 shows an alternative connector profile having an unbiased leading end 191, a forward flaring leading conical bore 192, and a backward rapidly expanded conical bore 193 in the trailing end designed for interconnecting conventional IV access ports with filled cartridges or syringes of volume capacity greater than 2.0 to 3.0. mL. This profile is unlikely to prove advantageous for users or patients,

FIG. 20 shows an alternative connector profile having a biased and flaring leading bore 201 and a sharply flaring trailing bore 202. Note that the inserted cylinder 203 of relatively small volume capacity has a very short surface in the trailing bore 202 for wedge impaction WI of the leading end of the cylinder 203 into the bore. The elastomeric cap 204 snugly inserted into the leading bore 201 is penetrated by a relatively short needle 205.

FIG. 21 shows an alternative connector profile having a cylindrical and biased leading bore 211, a gently sloping trailing bore 212, and an intervening. section 213 more sharply coned to rapidly reduce the diameter of the axial bore. Note that the inserted cylinder 214 of relatively large volume capacity has a longer surface in the trailing bore 212 for wedge impaction WI of the leading end of the cylinder 214 into the bore. The elastomeric cap 215 snugly inserted into the leading bore 211 is penetrated by the exposed bevel and shank of a longer needle 216.

Such variations in the profile-of the hollow connector, along with variation in the length of the exposed bevel and shank of the needle attached to a cylindrical cartridge or a syringe will permit the connector to accommodate a wide variety of hollow cylinders of differing lengths and volume capacities with a limited number of profiles wherein minor differences can be rectified by varying the length of the exposed bevel and shank of the attached needle. Never-the-less, the shorter the exposed parts of the needle, the likelihood of bending will be minimized and the likelihood of central axial penetration of the elastomeric cap in the leading bore will be maximized by appropriate coning of the trailing bore. Functionally, the more gentle the slope inside a hollow plastic cone, the more accurate will be central axial guidance of an inserted rigid cylinder and the more firm will be a tight wedge impaction at the point where the external diameter or circumference of the rigid cylinder exceeds the internal diameter of the hollow cone. In addition, making the hollow conical connector less rigid in substance than that of the inserted cylinder will increase the area of circumferential contact and, thus, increase the strength of the wedge impaction, such that twisting, as well as traction, is necessary to break the impaction.

As illustrated in FIGS. 1-8, during use of the pre-filled cartridge with a permanently affixed needle for IV port injection into an IV access system connected into the blood vessel of a patient, the user performs the following maneuvers in sequence. First, the user inserts the cartridge into the trailing end of a sterile connector to the point of wedge impaction. The needle scabbard can be removed before or after this maneuver, optionally after cartridge insertion, because an externally sterile needle scabbard may prevent fluid leakage from the cartridge. If the needle scabbard is removed before insertion of the cartridge into the connector, the external surfaces of the scabbard need not be sterile. Second, the user inserts the infusion limb of the IV access port into the leading bore of the connector to the point that the elastomeric needle cap has been sufficiently penetrated by the leading bevel and shank of the cartridge-attached needle. Third, the user long axially compresses the cartridge toward the infusion limb to be sure that connections and wedge impaction inside the connector are tight and secure. Fourth, the user gives the prescribed dose of liquid medication into the port by inserting a plunger to activate the cartridge piston. Fifth, the user sequentially removes the cartridge plunger and removes the IV infusion port from the leading end of the connector, grasping the connector to do so. Finally, the user discards the connector containing a safely recessed needle and emptied cartridge into an appropriate waste container.

As illustrated in FIGS. 8-16, use of the connector with a syringe having an attached needle is similar, but differs in that needle scabbard must be removed to fill the syringe with fluid medication by traction on a plunger permanently attached to the syringe piston. Therefore, immediately after filling, the syringe and attached needle should be inserted into the connector. The remaining maneuvers are the same, except that the plunger/piston are finally discarded, along with the emptied syringe.

In actual use of the connector with either application, it will be found that insertion of the needle bevel and shank through the elastomeric cap of the infusion port provides remarkably firm axial stabilization of the inter-connected parts. Such stabilization is owing to the fact that elastomeric compression of the cap around the contained needle shank hinders movement in the central axis.

It should be added that the needle hubs in some cartridges and the Luer-Loks in some syringes have external diameters equal to or greater than the external diameter of the cylindrical barrels. Therefore, the greatest external diameter must be taken into account when calculating the precise point in the connector bore where the inserted cartridge or syringe will wedge impact in order to assure optimal safe positioning of the needle bevel and shank before and after penetration of the elastomeric cap.

Employment of the connector with an inserted pre-filled cartridge is relatively efficient, because the user does not spend valuable time filling the cylindrical device. Employment of the connector with a standard syringe/needle is less efficient, but probably less expensive currently. Employment of either application is more efficient than use of a needleless IV access port, because the same needle is used to fill a syringe and to give the fluid injection into the port, and the handling of needle hubs to remove needles or replace needles with blunt cannulae is eliminated.

Finally, each kind of connector application should prove less costly and less hazardous for patients, as well as healthcare workers, because almost constant shielding of the needle within sterile confines before and after use will minimize risks for contamination of injections and accidental needlesticks more or less simultaneously

Cogent Medical References

1. Hannigan P, Shields J W. Handwashing and use of examination gloves. Lancet 1998;351: 571.

2. Danzig L E, Short I J, Collins K, Mahoney M, et al. Bloodstream infections associated with a needleless intravenous infusion system in patients receiving home infusion therapy, JAMA 1995; 273: 1862-1864.

3. Shields J W. Patient versus healthcare worker risks in needleless device infusion systems. Infect Control Hosp Epidemiol 1998; 19: 86-87.

4. Bryce E A, Ford J, Chase L, Cataldo D T. Sharps injuries: defining prevention priorities. Am J Infect Control 1999; 27: 447-452.

5. Russo P L, Harrington G A, Spelman D W. Needleless systems: A review. Am J Infect Control 1999; 27: 431-434.

6. Shields J W. Patient hazards of needleless systems. Am J Infect Control 2000; 28: 321-322.

7. Kellerman S, Shay D K, Howard J, et al. Bloodstrean infections in home infusion patients: the influence of race and needleless intravascular access devices. J Pediatr 1996; 129: 711-717.

8. Cookson S T, Ihrig M, O'Mara E M, et al. Increased bloodstream infection rates in surgical patients associated with variation from recommended use and care following implementation of a needleless device. Infecr Control Hosp Epidemiol 1998; 19;23-27.

9. McDonald L C, Baneijee S N, Jarvis W R. Line-associated bloodstream infections in pediatric intensive-care-unit patients associated with a needleless device and intermittent intravenous therapy. Infect Control Hosp Epidemiol. 1998; 19:772-777.

10. Do A N, Ray B J, Baneijee S N, et al. Bloodstream infection associated with needleless device use and the importance of infection control practices in the home health care setting. J Infect Dis 1999; 179: 442-448.

11. Raad, I. Intravascular catheter-related infections. Lancet 1998; 351: 522-527.

12. Wenzel R P, Edmond M B. The impact of hospital-acquired blood stream infections. CDC—Emerging Infectious Diseases—Special Report. 2001; 7(2): 1-6

PRIOR ART CITED

| | | |
|---|---|---|
| 4,998,925 | Mar. 12, 1991 | Al Sioufi |
| 5,558,649 | Sep. 24, 1996 | Shields |
| 6,245,056 | Jun. 12, 2001 | Walker |

Therefore, I claim:

1. A connector device operable for transferring a sterile fluid medication housed within a cylindrical container having a sharp hollow needle attached thereto, into an access port on an intravenous (IV) infusion system, said connector device enabling the transfer of medication from within the container into said access port having an infusion limb with a needle-penetrable cap attached thereto, the connector device comprising a hollow tubular structure having a leading cylindrical or conical axial bore dimensioned to receive and snugly hold a portion of the infusion limb of the access port in a fixed recessed location; a conical trailing bore dimensioned to receive and wedge impact a portion of the medication container having said sharp hollow needle attached thereto; and a central axial bore length between said fixed recessed location of said portion of said infusion limb and said wedge impaction of said portion of said medication container dimensioned such that the sharp tip of said sharp hollow needle is constantly recessed safely in said leading cylindrical or conical bore of the connector device after insertion, during use and after removal of said portion of said infusion limb of said access port from said leading axial bore of the connector device.

2. The connector device of claim 1 wherein a leading portion of said medication container becomes wedge impacted when inserted into the trailing conical bore and permits flow from the medication container into said IV access port after the attached sharp hollow needle penetrates said needle-penetrable cap on said infusion limb inserted into said fixed recessed location in said leading axial bore of the connector device.

3. The connector device of claim 1, wherein the hollow tubular structure is optically transparent and is less rigid than said container.

4. The connector device of claim 1, wherein said leading axial cylindrical or conical bore is cut off on a bias or flared to facilitate insertion of a needle-penetrable elastomeric cap on a trailing part of said infusion limb on said IV infusion system inserted into said fixed location inside said leading axial bore of the connector device.

5. The connector device of claim 1, wherein addition of trailing flanges, changing slopes of said leading or trailing axial bores, and/or varying the length of the bevels and shanks on said sharp hollow needle can make a limited variety of such connector devices uniquely applicable to a wide variety of cylindrical containers of varying sizes, fluid volume capacities and hub-attached needle lengths.

6. The connector device of claim 1, wherein an inserted hollow cylindrical container having a leading hub-attached sharp hollow needle and trailing sliding cylinder piston further comprise a variety of configurations with differing combinations of parts and fluid volume capacities, including:
a) a cartridge pre-filled with sterile liquid medication expelled under the aegis of a trailing sliding piston without a permanently attached cartridge plunger; a permanently hub-attached sharp hollow needle for draining said sterile liquid medication into a Y-infusion port on an IV infusion system; and wherein said bevel and shank of the sharp hollow needle are always safely recessed and confined within said leading axial bore of the connector after cartridge insertion, during fluid injection, and during safe disposal after the infusion limb and needle-penetrable elastomeric cap on said Y-infusion port are withdrawn from said heading axial bore of the connector device; a conventional Luer-Lok syringe having a plunger permanently attached to a sliding syringe piston;
b) a Luer-Lok attached to the hub of the leading sharp hollow needle used for filling a Luer-Lok syringe with sterile liquid medication and injecting said sterile liquid medication into a straight infusion port on said IV infusion system; and wherein said bevel and shank of the sharp hollow needle are always safely recessed and confined in said leading bore of the connector during and after intended use, as described in (a); and
c) either of the above, wherein this simple, efficient, and generic connector device with disclosed variations is alternatively attachable via a similar needle-penetrable access port to an arterial catheter line, a spinal catheter assembly, a dialysis system, an implanted port for infusion of medications or the like.

7. A connector device operable for transferring a sterile fluid medication housed within a container having a hollow bore needle attached thereto, into a conventional intravenous (IV) access port on an intravenous infusion system, said connector device enabling the transfer of medicament from within the container into a Y-shaped IV access port having an infusion limb with a needle-penetrable septum attached thereto without exposing the user to accidental needle-stick injury, the connector device comprising a hollow tubular member having a length, a conical leading axial bore dimensioned to receive and wedge impact a portion of the infusion limb of the IV access port and a conical trailing axial bore dimensioned to receive and wedge impact a portion of the medication container having said hollow bore needle attached thereto wherein both said leading and trailing axial bores taper inwardly toward the center of said length.

8. The connector device of claim 7 wherein when said portion of said medication container is received within said trailing conical bore, the hollow bore needle punctures the septum on the infusion limb of the IV access port, the hollow bore needle thereafter providing a conduit for fluid transport between the medication container and the IV access port.

9. The connector device of claim 7 wherein said length of said connector device is greater than the sum of the length of the hollow bore needle and the length of said portion of the medication container received within said trailing axial bore such that when the connector device is removed from the IV access port, the medication container remains wedge impacted within the trailing bore and the sharp point of the hollow bore needle is protectively housed within the axial bore of the connector device thereby preventing accidental needlestick injury.

10. The connector device of claim 8 wherein said length of said connector device is greater than the sum of the length of the hollow bore needle and the length of said portion of the medication container received within said trailing axial bore such that when the connector device is removed from the IV access port, the medication container remains wedge impacted within the trailing bore and the sharp point of the hollow bore needle is protectively housed within the axial bore of the connector device thereby preventing accidental needlestick injury.

11. A connector device operable for transferring a sterile fluid medication housed within a container having a hollow bore needle attached thereto into a conventional intravenous (IV) access port on an intravenous infusion system, said connector device enabling the transfer of medicament from within the container into a tubular IV access port having a proximal end with a needle-penetrable septum attached thereto and an annular, elastically compressible member overlying said proximal end, the connector device comprising a hollow tubular member having a length, a leading axial bore dimensioned to receive and releasably attach to said proximal end of the infusion limb of the IV access port, and a conical trailing axial bore dimensioned to receive and wedge impact a portion of the medication container having said hollow bore needle attached thereto wherein said trailing axial bore tapers inwardly toward the leading axial bore.

12. The connector device of claim 11 wherein when said portion of said medication container is received within said trailing conical bore, the hollow bore needle punctures the septum on the infusion limb of the IV access port, the hollow bore needle thereafter providing a conduit for fluid transport between the medication container and the IV access port.

13. The connector device of claim 12 wherein when the medication container is wedge impacted within said trailing conical bore, the sharp tip of the hollow bore needle is contained within said leading axial bore.

14. The connector device of claim 7 wherein the connector device has unitary construction.

15. The connector device of claim 11 wherein the connector device has unitary construction.

16. The connector device of claim 7 wherein the connector device has a beveled leading end.

17. The connector device of claim 11 wherein the connector device has a beveled leading end.
